(19) United States
(12) Patent Application Publication
(10) Pub. No.: US 2005/0183726 A1
(43) Pub. Date: Aug. 25, 2005

(54) Device and method for the targeted supply of oxygen to the location of respiratory organs, in particular within aircraft

(76) Inventors: Hans-Juergen Heinrich, Hamburg (DE); Robert Surma, Hamburg (DE)

Correspondence Address: OSTROLENK FABER GERB & SOFFEN, 1180 AVENUE OF THE AMERICAS, NEW YORK, NY, 100368403, US

(21) Appl. No.: 11/019,373

(22) Filed: Dec. 22, 2004

(30) Foreign Application Priority Data
Dec. 24, 2003 (DE) 103 61 271.8

(51) Int. Cl. A62B 7/04; A61M 16/00

(52) U.S. Cl. 128/204.29; 128/205.25

(57) ABSTRACT

The invention relates to a device and a method for the targeted supply of oxygen to the location of respiratory organs, in particular within aircraft cabins or similar. In the conventional way, this supply was previously provided by the means of breathing masks, whereby the mask had to be actively placed over the nose and/or mouth by the people to be supplied or with the help of other people. The disadvantage of this is that incorrect application can arise. Moreover, the wearing of the breathing masks over an extended period of time can be uncomfortable. By means of the form of a device in accordance with the invention, ie. where the line itself and the outlet opening are the means for the supply of oxygen to the location of the respiratory organs, the unreliable breathing masks which can also get in the way no longer have to be worm. The direct supply of oxygen from the outlet opening can take place without the person being supplied having to take any action so that faulty application is effectively prevented.

### FIELD OF THE INVENTION

The present invention relates to a device and method for the targeted supply of oxygen to the location of respiratory organs, in particular within aircraft cabins or similar.

### TECHNOLOGICAL BACKGROUND

The provision of passengers and the cabin and flight crew with additional oxygen is necessary, for example, if there is a drop in pressure in the aircraft cabin. For this purpose, the oxygen must reach the area where the respiratory organs are located or the respiratory organs. It can also be necessary, when preparing certain flight operations such as, eg. work on an open hatch of an aircraft at great heights, to significantly increase the concentration of oxygen in the blood. In such instances it is necessary to increase the supply of oxygen over an extended period of time, eg. 1 hour. With this so-called “pre-breathing”, people can be prepared for special circumstances.

Established devices of this type are equipped with so-called breathing masks as a means of directing the oxygen from the outlet opening to the respiratory organs. These breathing masked are connected to the lines with tube-like elements, and in this way allow the oxygen to flow indirectly to the respiratory organs. It is indirect because additional devices are required to increase the concentration of oxygen in the area where the respiratory organs are located, whereby oxygen is directed from the line to the respiratory organs via the breathing mask. For this, the breathing mask should be positioned in the nose and/or the mouth area or placed over the nose and/or mouth. If pressure drops, the breathing masks, which are normally located above each seat in covered hollows, boxes, containers or similar, drop down from these. At the same time or very soon afterwards, the line and/or the means for supplying the oxygen is opened. By the means for conveying the oxygen from the supply container to the outlet opening, which can be in the form of a pump or other for bringing about a fall in pressure, the oxygen flows out of the line and through the breathing mask into the respiratory organs. Each individual person must actively put their breathing mask on in order to be supplied with oxygen. If a person is preparing for particular flight operations or special circumstances, he must wear a breathing mask for an extended period of time.

The disadvantage, however, of these established devices and methods is that incorrect application of the breathing mask, which is particularly likely in stressful conditions or with children, will lead to insufficient supply of oxygen. Moreover, the masks falling from the hollows can also have a negative psychological effect because the necessity to be supplied with additional oxygen implies an emergency. In addition, it is uncomfortable wearing a breathing mask over an extended period of time and it also limits the freedom of movement when carrying out special flight operations.

### SUMMARY OF THE INVENTION

According to an exemplary embodiment of the present invention, an oxygen supply device is provided, comprising a supply container for oxygen, at least one line connected to the supply container, a means for conveying the oxygen from the supply container to an outlet opening in the line and a means for taking the oxygen from the outlet opening to the respiratory organs. Moreover, according to an exemplary embodiment, a method for the targeted supply of oxygen to the location of respiratory organs, in particular within aircraft cabins or similar is provided, comprising the following steps: conveyance of the oxygen from a supply container by means of at least one line to an outlet opening of this line, release of the oxygen through the outlet opening, and supply of the oxygen to the respiratory organs. This may allow for a simple and reliable method and device.

According to another exemplary embodiment, the line itself and the outlet opening are the means for supplying the oxygen to the location of the respiratory organs. This means that additional devices, such as eg. masks, are no longer required. The person being supplied with oxygen no longer has to take any action, and this rules out all risk of incorrect application. In other words, the supply of additional oxygen takes place without the person, namely the passenger, the flight personnel or similar, having to do anything, and this is a great advantage in situations of stress. The line as a means of supply may guarantee—unlike indirect oxygen supply—a direct supply to the respiratory organs. Moreover, according to an exemplary embodiment of the present invention, there is no need for additional devices, which makes it possible to offer heightened comfort because breathing masks do not have to be worn, and this is may be advantageous if “pre-breathing” is necessary over an extended period of time. No less importantly, an embodiment of the invention is believed to facilitate “imposed breathing”, namely the supply of oxygen to the respiratory organs of animals so that, when transporting animals in the cargo area of an aircraft, the animals can also be supplied with oxygen if pressure drops. This is, of course, not possible with breathing masks.

The oxygen may be conveyed or supplied as a directed free jet from the outlet opening to the location of the respiratory organs. The supply of oxygen by means of the free jet ensures that sufficient oxygen enters the area where the respiratory organs are located without the person being supplied having to take any action. In this way one can ensure a stress-free and reliable supply of oxygen, in particular in emergencies or during special flight operations. The free jet directly supplies the location of the respiratory organs without any elements that could be awkward or likely to cause problems.

A directable and/or adjustable nozzle and/or tube element may be positioned beneficially in the area surrounding the outlet opening. This means, on the one hand, that the direction of the free jet can be determined so that, eg. it can be adapted to suit the body size of the person. On the other hand, it means that the required stream of oxygen is adjustable by using the pressure regulator in the line so that it is possible to adapt individually to the circumstances and requirements in question.

In another exemplary embodiment of the invention, the speed and oxygen concentration of the free jet is adjustable with the help of the pressure regulator. On the one hand, this may allow to provide for a sufficient, directed supply of oxygen, and on the other hand it may ensure that consideration of the well-being of the person being supplied is prevented.

A heating element is preferably provided in the area around the line and/or the nozzle and/or tube element. By heating, it is possible for the stream of oxygen to show an increase in specific volume so as to prevent the oxygen from sinking into the surrounding air or to ensure that it sinks more slowly.

In another exemplary embodiment of the invention, the device is designed and/or adapted to be mobile and in such a way that it can be carried as a unit on a person's body. For one thing, this increases flexibility, particularly with regard to freedom of movement, because the oxygen supply does not depend upon location.

Moreover, a method with the aforementioned steps is provided where the oxygen is supplied directly from the line or the outlet opening to the respiratory organs. By means of direct supply—without the intervention of additional components—a simple and highly reliable supply may be allowed because oxygen is supplied without the person being supplied having to do anything. Moreover, the level of comfort may be improved because breathing masks, which offer indirect supply, do not have to be worn.

The oxygen is beneficially supplied as a directed free jet from the outlet opening to the or to the location of the respiratory organs. Because the oxygen is supplied directly from the outlet opening into the environment, (the aircraft cabin for example), the procedure is particularly easy and stress-free, also because there is no longer the psychological factor of an emergency situation being announced by the dropping down of breathing masks.

### DETAILED DESCRIPTION OF EXEMPLARY EMBODIMENTS

Embodiments which are particularly favoured and the principle of the method are more clearly illustrated by the attached drawing. In the drawing:

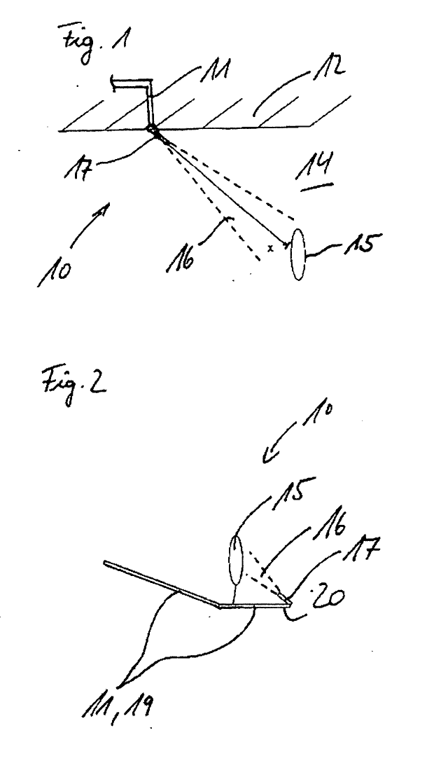

FIG. 1 shows parts of a schematic device with a nozzle and tube element used to direct a free jet of gaseous oxygen,

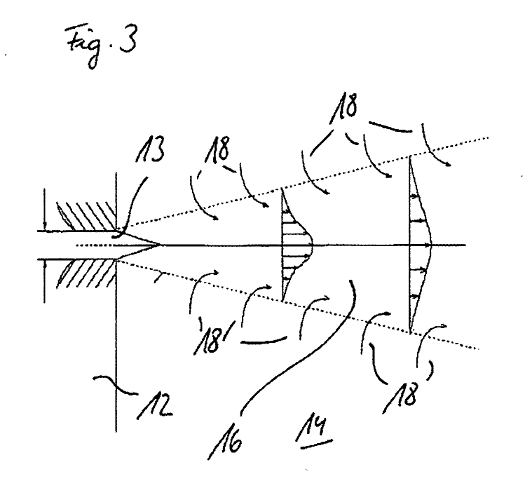

FIG. 2 shows parts of a schematic device with a support element specially to be worn on the body, and

FIG. 3 shows a schematic representation of the spreading of the free jet.

The forms of the devices shown are used to provide passenger and cabin and flight crew with additional oxygen.

In FIG. 1, a device 10 and its relevant parts are shown which supply passengers with additional oxygen. Because the basic structure of such devices 10 is known, a detailed representation was not provided. The device 10 includes a container (not shown) to receive and store the gaseous oxygen. The container can be positioned wherever you like. At least one line 11 (as indicated) runs from the container in the direction of an area from which the line 11 and the oxygen conveyed by the line emerges. The or each line 11 is preferably located in a covered element 12 above a seat in an aircraft cabin. In the area around an outlet opening 13 the line 11 leads to the space 14 to be supplied with oxygen. The line 11 and the outlet opening 13 alone provide the means for supplying oxygen to the location of the respiratory organs 15 whereby a free jet 16 of oxygen emerging from the outlet opening spreads into the area where the respiratory organs 15 are located.

The space 14 can be an aircraft cabin, a cargo space within the aircraft or any other space. If the oxygen supply is released, oxygen is released from the line 11 at the outlet opening 13, whereby the oxygen is conveyed by means of a device (not illustrated), which eg. creates a difference in pressure between the container and the line 11 on the one hand and the environment, eg. the space 14. The outlet opening 13 and/or the line 11 itself can be closed and then opened again by conventional elements which are not illustrated. These elements are preferably joined to a control and/or regulation unit. The elements can also, however, be adjusted by the pressure regulator in the line 11. The outlet opening 13 has a defined cross-section which can be different dependent upon the requirements. The cross-section is preferably circular so that a conical free ray 16 can emerge from the outlet opening 13.

The respiratory organs 15 are generally located or positioned at a distance X from the outlet opening 13. The basis for determining the distance X are the average values of people relating to their size. Particularly favoured are distances X in an area of approx. 0 to 0.7 m between the exit cross-section and the respiratory organs 15. Other distances are, however, possible. The distance X and the area between the outlet opening 13 and the respiratory organs 15 are free from components of the device 10 itself so that the free ray 16 can spread out without any hindrances.

A nozzle and/or tube element 17 (see FIG. 1 in particular) should preferably be positioned in the area of the outlet opening 13. The nozzle and/or tube element 17 is designed to be directable and/or adjustable and in particular variable so that the free ray 16 can be positioned with regard to target and direction. After the free ray 16 has been emitted from the outlet opening 13 or the nozzle and/or tube element 17, the free ray (or flow) 16 will hit the ambient air 18 and mix with the same. The free ray 16 is calculable on the basis of a constant impulse stream. This means that, as well as the speed of the free ray 16 and the diffusion, the oxygen concentration of the free ray 16 can also be calculated in relation to the distance X. Refer here to the whole of the method suggested by Ricou and Spalding (see eg. F. P. Ricou, D, B, Spalding: Measurements of entrainment by axisymmetrical turbulent jets, J. Fluid Mech. 11 (1961), pages 21 to 32).

FIG. 3 shows the diffusion of an isothermal gaseous free ray in a gaseous atmosphere. The free ray 16 flows out of the outlet opening 13 of the line 11 or the nozzle and/or tube element 17. As a result of the mixing of the oxygen with the ambient air 18, the concentration and the speed of the free ray 16 decrease as the distance from the outlet opening 13 becomes greater. At the same time, the free ray 16 broadens out. The free ray 16 calculation is made in accordance with the method specified above. The speed of the free ray 16 in front of the respiratory organs 15 is preferably between approx. 1 m/s/s and 10 m/s. Below the lower limit, there is the risk of the free ray 16 disintegrating before the additional oxygen has reached the respiratory organs. If the upper limit is passed, the flow of the free ray 16 will, under certain circumstances, cause problems. The size of the stream flowing out of the outlet opening 13 provides the required and/or necessary concentration of oxygen in relation to distance X between the outlet opening 13 and the respiratory organs 15. The local oxygen concentrations should ideally be set at between approx. 20% and 80% in relation to the distances X from 0 m to 0,7 m. The area details are variable, however, so that that other concentrations can also be set, in particular where the distances are greater.

FIG. 2 shows another exemplary embodiment of the device 10. Here, the device 10 is shown as a mobile unit. The principle of the action-free local oxygen supply for the person to be supplied corresponds to that already described. The line 11 is designed and arranged in such a way that the outlet opening 13 points in the direction of the respiratory organs 15. The unit can, for example, can be in the same form as the oxygen bottles well-known to diving sports, and carried on the back. The line 11 is made from flexible tubing and set in place using fixing elements 19, whereby an end-piece 20 of the line 11 or a nozzle and/or tube element 17 positioned on an end-piece 20 is fastened to the head area similarly to a head-set or similar.

In another exemplary embodiment, in addition to the version shown in FIG. 1, there is a hood-type element positioned in the area of the covering element 12. The element, at least partially, is positioned next to an area of air into which oxygen is introduced from above and at a slow speed by means of a free jet. By heating it can, if so required, be ensured that the ambient air enriched with oxygen in the area surrounding the element is detained so that additional oxygen stays in the head area of a person sitting beneath the element for an extended period of time. The element is positioned in such a way that it allows the freedom of movement required, and in particular, freedom of the head. For this, the element can be, eg. rotatable or hinged so that it is only positioned above the area to be supplied if so required.

In the following, an exemplary embodiment of the method of the present invention is described in greater detail using the exemplary embodiment of the device in accordance with FIG. 1:

If pressure drops in the aircraft cabin, the outlet opening 13 opens so that stored, gaseous oxygen flows out of a container and the line 11. The outlet opening 13 and the nozzle and/or tube element 17 is directed so that the free jet 16 of oxygen which forms goes straight to the respiratory organs 15 or at least to the area where the respiratory organs 15 are located. This basic or additional supply of oxygen takes place without the person to be supplied taking any action whatsoever. The oxygen is quasi issued from the line 11 and the outlet opening 13 directly into the space 14. The free jet 16 reaches the respiratory organs 15 at a speed which is preferably approx. 10 m/s. If the oxygen concentration supplied is not sufficient, heightened concentration can be achieved simply by reducing the distance, eg. by inclining the head. In addition, the oxygen can be heated, whereby the specific volume changes, or more particularly increases. In this way, the difference in comparison to the specific volume of the ambient air is reduced so that the oxygen remains for a longer time in the upper area of the head.

Other exemplary embodiments shown and not explicitly explained follow the same principle. With the version with the mobile unit, the person wearing the device 10 can move freely and yet still benefit from an increase in oxygen content in the blood parallel to this. The additional supply can even continue to be provided during the flight operation so that a sufficient supply is ensured. The positioning of the device 10 on the body means that the device 10 quasi follows the movements of the person being supplied. As well as for use in aircraft, the device can also be used in other areas where an additional supply of oxygen is required, for example in areas where tanks are being cleaned.

1. Device for a targeted supply of oxygen to an area where respiratory organs are located, particularly within aircraft cabins or similar, comprising
a supply container for oxygen;
at least one line connected to the supply container, the at least one line having an outlet opening;
a conveyer for conveying the oxygen from the supply container to the outlet opening;
wherein the at least one line itself and the outlet opening are adapted for supplying the oxygen to the area where the respiratory organs are located.

2. The device of claim 1, wherein the oxygen is directed as a free jet from the outlet opening to the area where the respiratory organs are located.

3. The device of claim 1, wherein the area between the outlet opening and the respiratory organs is free from any components of the device itself.

4. The device of claim 1, wherein there is a distance between the outlet opening and the respiratory organs.

5. The device of claim 1, further comprising
a nozzle, which is at least one of directable and adjustable; and
a tube element;
wherein the tube element is positioned adjacent to the outlet opening.

6. The device of claim 2, wherein a speed of the free jet is variable.

7. The device of claim 2, wherein the free jet is conically shaped.

8. The device of claim 2,
wherein the free jet is gaseous; and
wherein the gaseous free jet and the diffusion of the same is calculable on the basis of
a constant impulse stream.

9. The device of claim 2, wherein the free jet is in a gaseous atmosphere.

10. The device of claim 5, further comprising
a heating element;
wherein the heating element is positioned adjacent at least one of the at least one line, the nozzle and the tube element.

11. The device of claim 1, wherein the device is arranged to be mobile in such a way that it can be positioned on the body of a person as a carryable unit.

12. The device of claim 11, wherein at least one line is arranged and positioned in such a way that the outlet opening points in the direction of the respiratory organs.

13. The device of claim 1, wherein a concentration of the oxygen can be adjusted directly in front of the respiratory organs.

14. Method for the targeted supply of oxygen to the location of respiratory organs, in particular within aircraft cabins or similar, comprising the steps of:
conveying oxygen from a supply container via at least one line to an outlet opening of the same;
outwards flowing of the oxygen from the outlet opening;
supplying the oxygen to the respiratory organs; and
supplying the oxygen directly from at lease one of the at least one line, and the outlet opening to the respiratory organs.

15. The method of claim 15, further comprising the step of directing the oxygen as a free jet from the outlet opening to the location of the respiratory organs or in the respiratory organs.

16. The method of claim 15, wherein the free jet reaches the respiratory organs at a speed of at least approx. 1 m/s, preferably, however, 10 m/s.

17. The method of claim 15, further comprising the step of:
broadening out the free jet as the distance from the outlet opening increases.

18. The method of claim 14, further comprising the step of:
adjusting a concentration to heighten a local oxygen concentration based upon a provision of a flow of oxygen.

19. The method of claim 18, further comprising the step of:
heating the oxygen of the flow of oxygen.

20. The method of claim 15, further comprising the step of:
calculating a diffusion of the gaseous free jet upon the basis of a constant impulse stream.

21. The method of claim 15, further comprising the step of:
ejecting the oxygen directly from the line or from the outlet opening into the environment.
